(19) United States
(12) Patent Application Publication
(10) Pub. No.: US 2022/0168453 A1
(43) Pub. Date: Jun. 2, 2022

(54) EXERCISE EQUIPMENT WITH ANTIMICROBIAL SUBSTANCE DISPOSED ON EXPOSED SURFACE

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 17/539,588

(22) Filed: Dec. 1, 2021

Related U.S. Application Data
(60) Provisional application No. 63/119,742, filed on Dec. 1, 2020.

(51) Int. Cl. A61L 2/18; A63B 21/078; A63B 21/00; A63B 21/072; A63B 21/062; A63B 22/06; A63B 22/02; A63B 22/00; A63B 22/04

(52) U.S. Cl. CPC A61L 2/18; A63B 21/078; A63B 21/4035; A63B 21/0726; A61L 2/186; A63B 22/06; A63B 22/02; A63B 22/0076; A63B 22/04; A63B 21/0628

(57) ABSTRACT

An exercise equipment comprises: an exposed surface; and a silane quaternary ammonium ion or salt thereof disposed on the exposed surface. The exercise equipment can further comprise a handle, wherein the handle comprises the exposed surface. The exercise equipment can further comprise a seat, wherein the seat comprises the exposed surface. The exercise equipment can further comprise a control panel, wherein the control panel comprises the exposed surface. The exercise equipment can further comprise a seatback, wherein the seatback comprises the exposed surface. The silane quaternary ammonium ion or salt thereof can comprise one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride. The exercise equipment can be any of a barbell, a plate-loaded machine, a workout bench, a medicine ball, a jump rope, an elliptical machine, a climbing machine, a stationary bicycle, a treadmill, and an elliptical machine.

### CROSS-REFERENCE TO RELATED APPLICATION

This application claims priority to and the benefit under 35 U.S.C. § 119(e) of U.S. Provisional Patent Application No. 63/119,742, filed on 1 Dec. 2020, entitled EXERCISE EQUIPMENT WITH ANTIMICROBIAL SUBSTANCE DISPOSED ON EXPOSED SURFACE, the entire disclosure of which is incorporated herein by reference.

### BACKGROUND OF THE DISCLOSURE

A person enjoys exercising and sometimes utilizes exercise equipment in a fitness center. However, there is a problem in that the exercise equipment sometimes harbors microbes, potentially deposited by one person and then transmitted to another person.

### SUMMARY OF THE INVENTION

The present disclosure addresses that problem by coating an exposed surface of the exercise equipment with a solution that includes silane quaternary ammonium ion or salt thereof. The solution dries, leaving the silane quaternary ammonium ion or salt thereof bonded to the exposed surface. The silane quaternary ammonium ion or salt thereof imparts antimicrobial properties to the exposed surface and thus the exercise equipment generally. In the event that one person deposits microbes onto the exposed surface, the silane quaternary ammonium ion or salt thereof destroys the microbe or reduces the ability of the microbe to reproduce, which reduces the ability of the exercise equipment to transmit the microbe to another person.

According to a first aspect of the present disclosure, an exercise equipment comprises: an exposed surface; and a silane quaternary ammonium ion or salt thereof disposed on the exposed surface. In embodiments, the exercise equipment further comprises a handle, wherein the handle comprises the exposed surface. In embodiments, the exercise equipment further comprises a seat, wherein the seat comprises the exposed surface. In embodiments, the exercise equipment further comprises a control panel, wherein the control panel comprises the exposed surface. In embodiments, the exercise equipment further comprises a seatback, wherein the seatback comprises the exposed surface.

In embodiments, the exercise equipment is a weight bench, and the weight bench comprises the exposed surface. In embodiments, the exercise equipment is a dumbbell, and the dumbbell comprises the exposed surface. In embodiments, the exercise equipment is a kettlebell, and the kettlebell comprises the exposed surface. In embodiments, the exercise equipment is a weight plate, and the weight plate comprises the exposed surface. In embodiments, the exercise equipment is a stack machine, and the stack machine comprises the exposed surface. In embodiments, the exercise equipment is a stationary bicycle, and the stationary bicycle comprises the exposed surface. In embodiments, the exercise equipment is a treadmill, and the treadmill comprises the exposed surface. In embodiments, the exercise equipment is a rowing machine, and the rowing machine comprises the exposed surface.

In embodiments, the exercise equipment is a stair machine, and the stair machine comprises the exposed surface. In embodiments, the silane quaternary ammonium ion or salt thereof comprises one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

According to a second aspect of the present disclosure, a method of treating an exercise equipment comprises: applying a solution comprising a silane quaternary ammonium ion or salt thereof to an exposed surface of an exercise equipment. In embodiments, the exercise equipment comprises a handle, and the handle comprises the exposed surface. In embodiments, the solution further comprises isopropyl alcohol. In embodiments, the solution further comprises hydrogen peroxide. In embodiments, the solution further comprises hypochlorous acid.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is a perspective view of a fitness center with exercise equipment, illustrating treadmills, stationary bicycles, among other things, each with an exposed surface;

FIG. 2 is a perspective view of another fitness center with more exercise equipment, illustrating a stack machine, a plate-loaded machine, a weight bench, a dumbbell, among other things, each with an exposed surface;

FIGS. 3-6 are perspective views of a kettlebell, a barbell with a weight plate and a collar, a rowing machine, and a head/neck harness, each with an exposed surface;

FIGS. 7-10 are perspective views of an elastic band, a yoke, a training sled, and a log, each with an exposed surface;

FIGS. 11-14 are perspective views of farmer's walk handles, a stone, a keg, and a power pin, each with an exposed surface;

FIGS. 15-18 are perspective views of a hammer, a stammer, sandbags, and a Bulgarian bag, each with an exposed surface;

FIGS. 19-22 are perspective views of parallel bars, a pull-up and dip belt, a peg board with pegs, and a pull-up bar, each with an exposed surface;

FIGS. 23-26 are perspective views of gymnastic rings, a weighted belt, ankle weights, and paralletes, each with an exposed surface;

FIGS. 27-30 are perspective views of push-up handles, a medicine ball, a Swiss ball, and a jump rope, each with an exposed surface;

FIGS. 31-33 are perspective views of a climbing rope, conditioning ropes, and a massage roller, each with an exposed surface;

FIGS. 34-37 are perspective views of a massage ball, an elliptical machine, a glider machine, and a climbing machine, each with an exposed surface;

FIGS. 38-39 are perspective views of a stair machine and a workout bench, each with an exposed surface; and

FIG. 40 is a perspective view of a method of treating the exercise equipment (including any of those of FIGS. 1-39) by applying a solution with a silane quaternary ammonium ion or salt thereof to the exposed surface of the exercise equipment to impart long-lasting antimicrobial protection.

### DETAILED DESCRIPTION

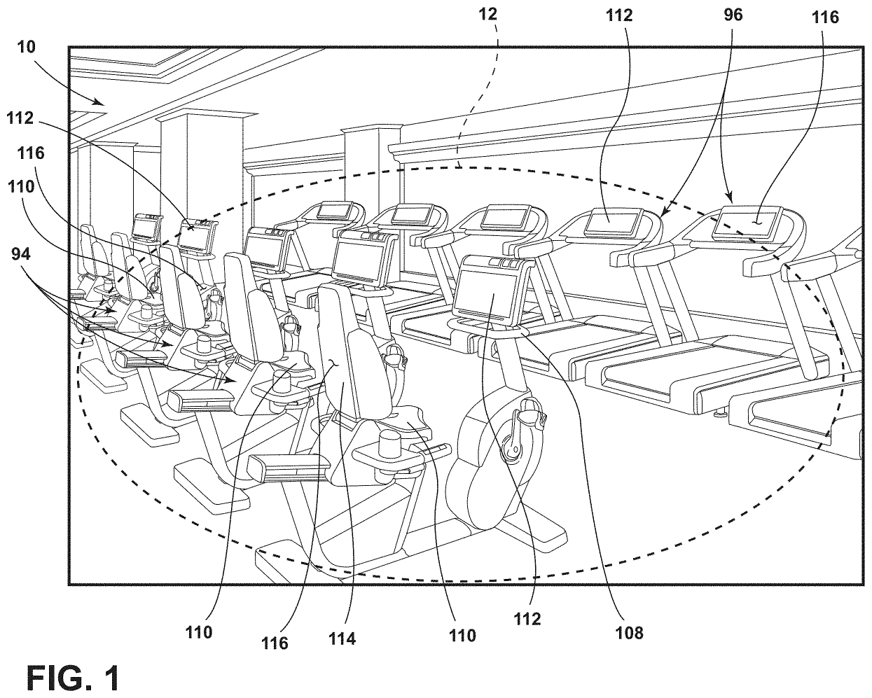

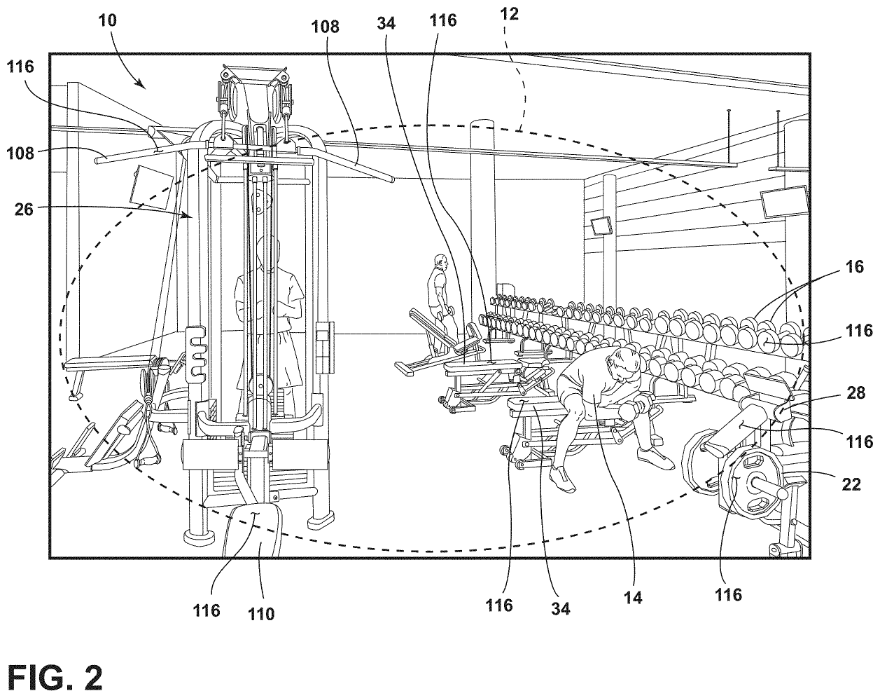

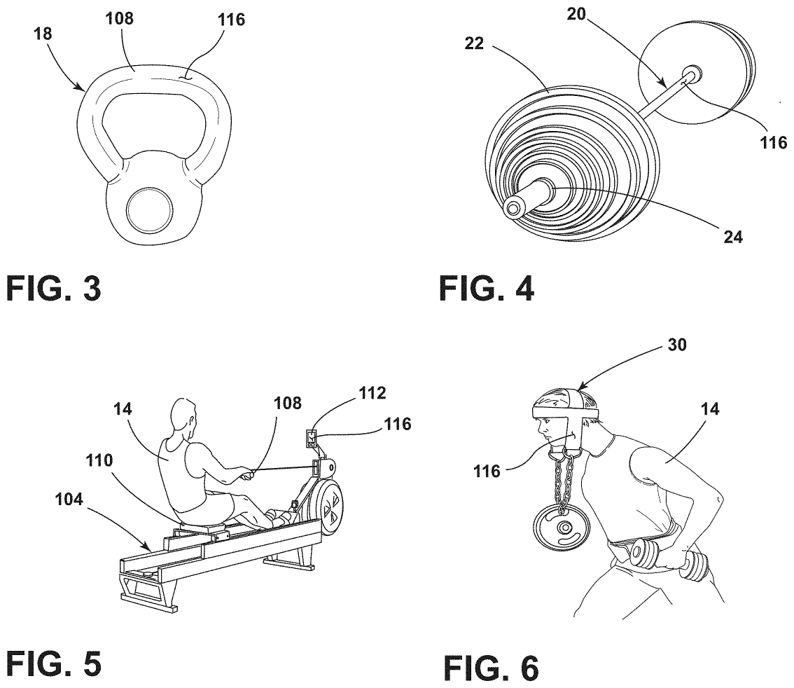

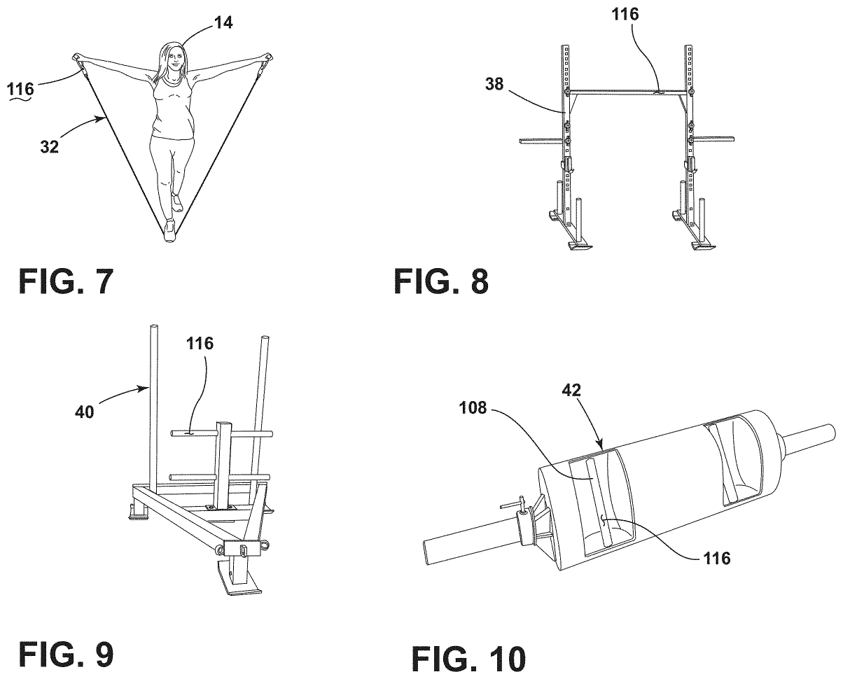

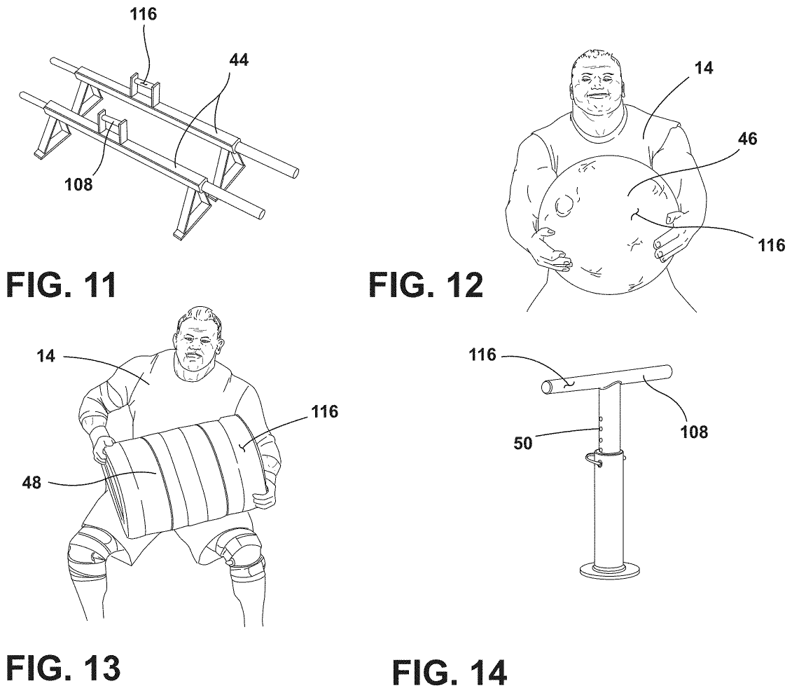

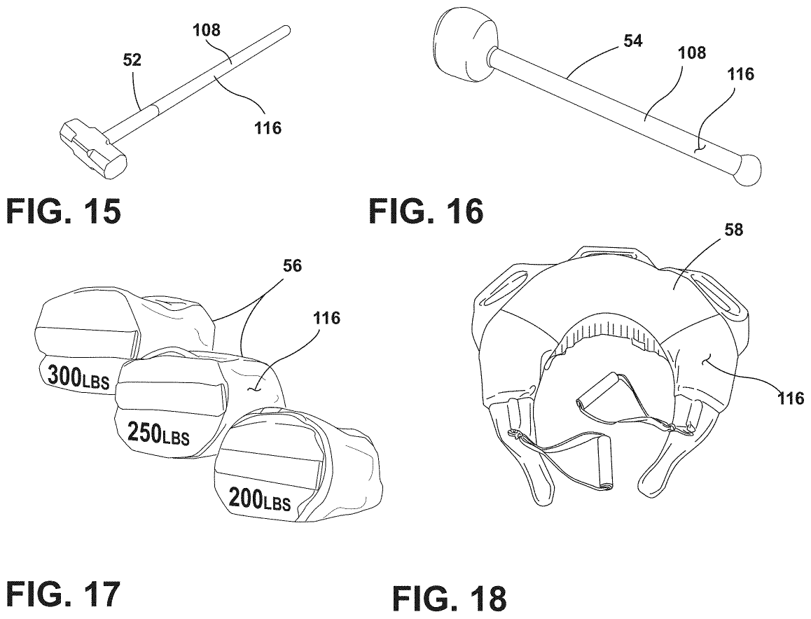

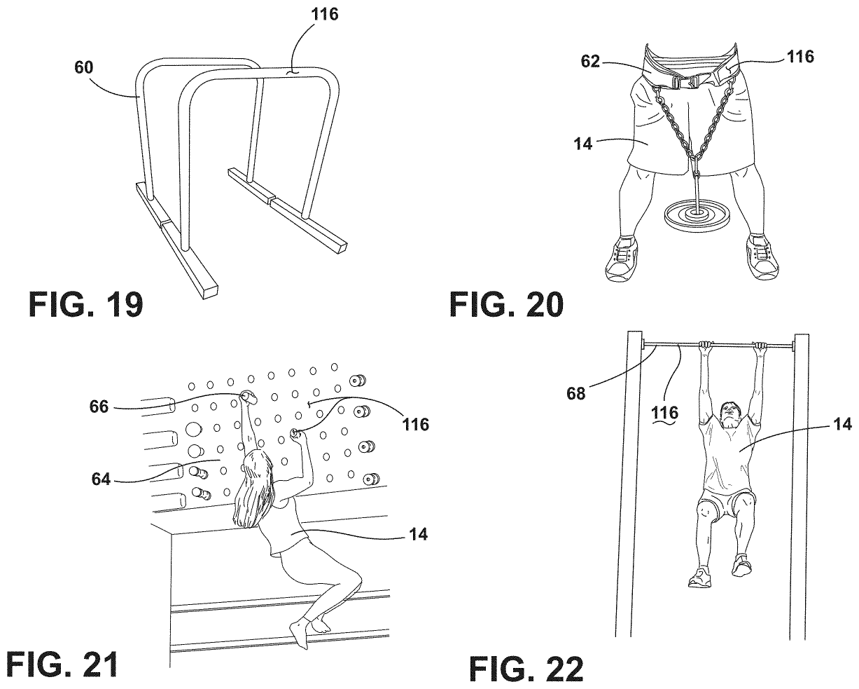

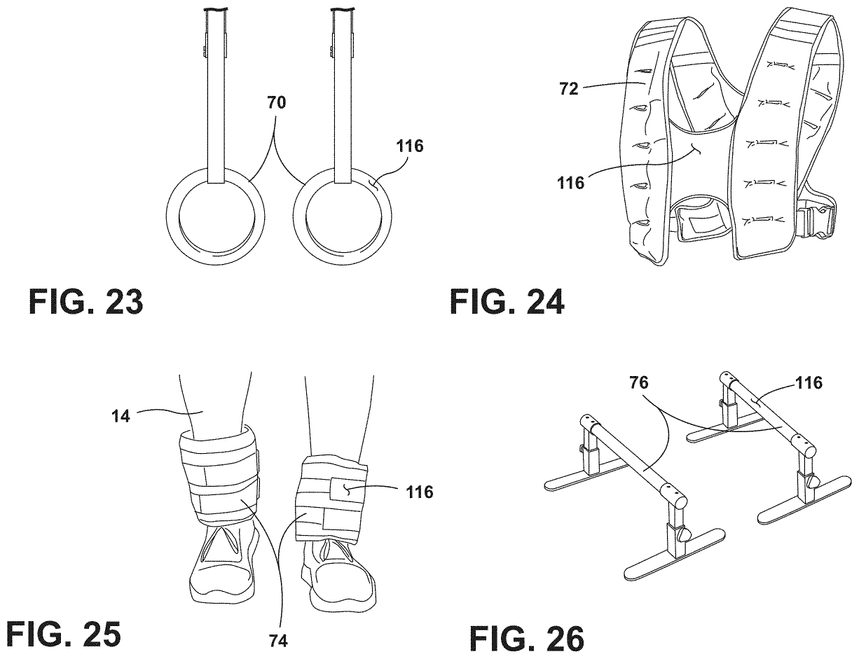

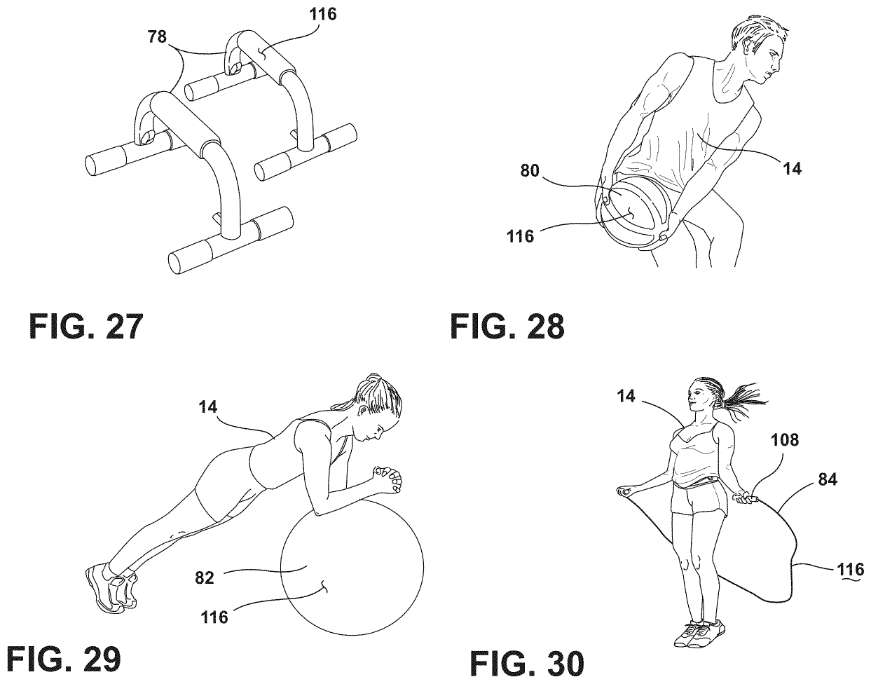

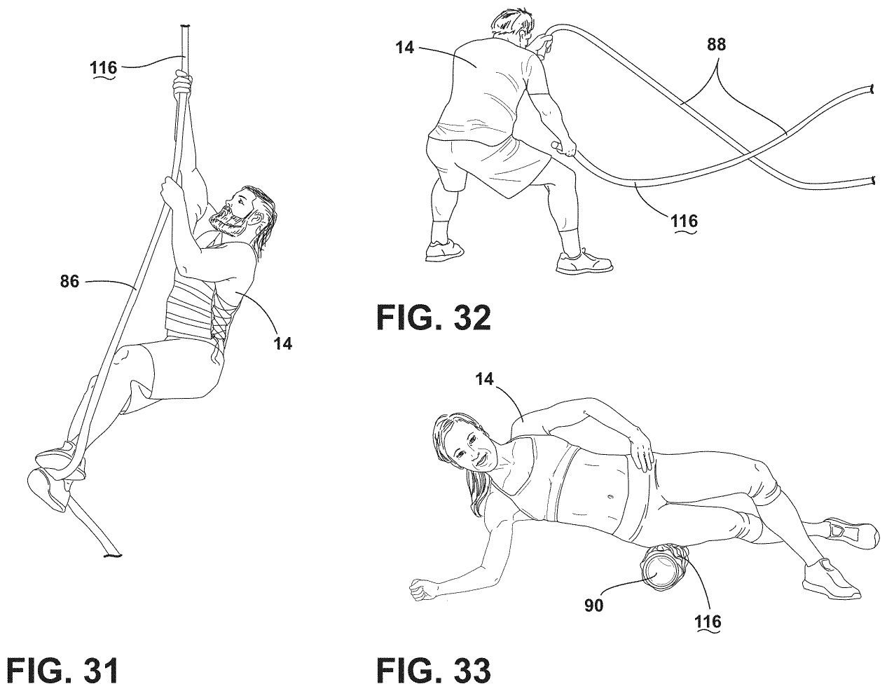

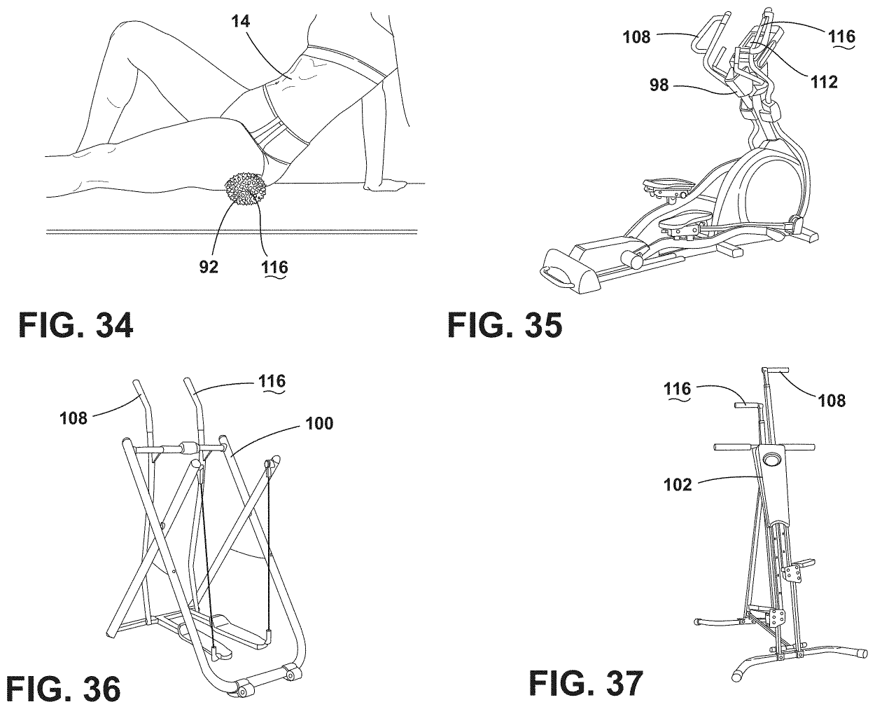

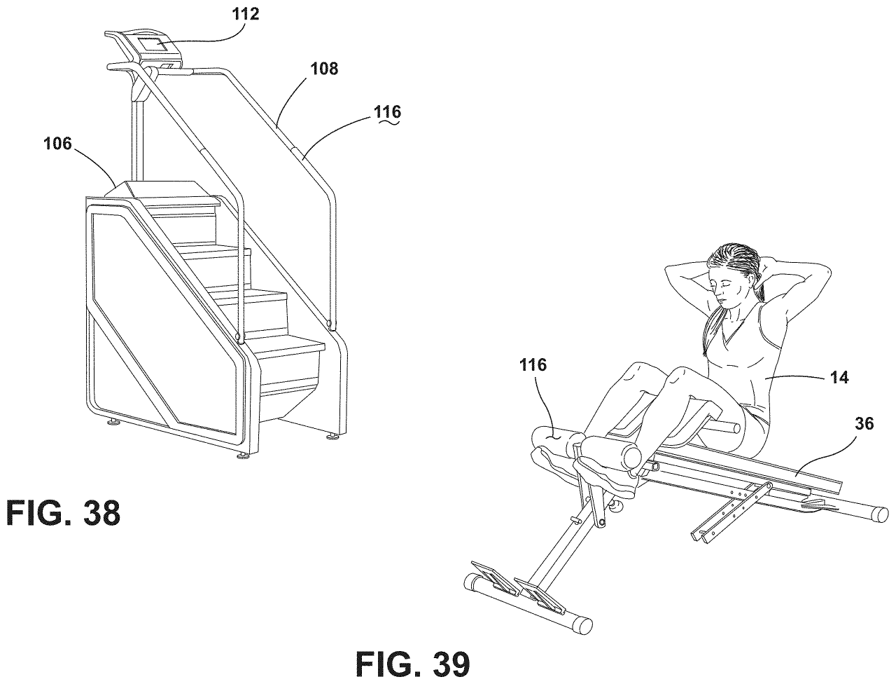

Referring now to FIGS. 1-39, a fitness center 10 includes exercise equipment 12. Exercise equipment 12 includes any apparatus that a person 14 uses to perform an exercise and any apparatus that the person 14 uses to enhance the exercise by increasing or decreasing resistance to movement. Exercise equipment 12 includes but is not limited to: (i) strength training equipment; (ii) strongman equipment; (iii) bodyweight training, calisthenics, and gymnastics equipment; (iv) myofascial release and recovery tools; and (v) exercise machines.

Strength training equipment includes but is not limited to: (i) free-weight training equipment such as a dumbbell 16, a kettlebell 18, a barbell 20, a weight plate 22, and a collar 24 (to secure the weight plate 22 onto the barbell 20); (ii) weight machines, such as a stack machine 26 and a plate-loaded machine 28; (iii) a head/neck harness 30; (iv) variable resistance training devices, such as an elastic band 32; and (v) a weight bench 34 and a workout bench 36.

Strongman equipment includes but is not limited to: a yoke 38, a training sled 40 (including a push sled and a pull sled), a log 42, farmer's walk handles 44, a stone 46, a keg 48, a power pin 50, a hammer 52, a slammer 54, a sandbag 56, and a Bulgarian bag 58.

Bodyweight training, calisthenics, and gymnastics equipment includes but are not limited to: parallel bars 60, a pull-up and dip belt 62, a peg board 64 and pegs 66, a pull- up bar 68, suspension training equipment including gymnastic rings 70, a weighted vest 72, limb weights 74 such as ankle and wrist weights, paralletes 76, push-up handles 78, a medicine ball 80, a Swiss ball 82, a jump rope 84, a climbing rope 86, and conditioning ropes 88.

Myofascial release and recovery tools include but are not limited to: a massage roller 90 and a massage ball 92.

Exercise machines include endless-path machines such as a stationary bicycle 94, a treadmill 96, an elliptical machine 98, a glider machine 100, a climbing machine 102, a rowing machine 104, and a stair machine 106.

In embodiments, the exercise equipment 12 includes a handle 108. For example, the stationary bicycle 94, the stack machine 26, rowing machine 104, the log 42, the treadmill 96, the elliptical machine 98, the glider machine 100, the climbing machine 102, and the stair machine 106, among others, include the handle 108. The person 14 grabs the handle 108, for example, to stabilize the person 14 using the exercise equipment 12. Other exercise equipment 12, such as the dumbbell 16, kettlebell 18, the hammer 52, and the slammer 54, all include the handle 108, which is where the person 14 holds onto the particular exercise equipment 12.

In embodiments, the exercise equipment 12 includes a seat 110. For example, the stack machine 26, the rowing machine 104, and the stationary bicycle 94, among others, include the seat 110. The person 14 sits on the seat 110 to use the particular exercise equipment 12.

In embodiments, the exercise equipment 12 includes a control panel 112. For example, the stationary bicycle 94, the treadmill 96, the elliptical machine 98, the rowing machine 104, and the stair machine 106, among others, include the control panel 112. The person 14 may, via the control panel 112, set a timer, view data relevant to an exercise session, set a level of resistance or speed, among other things.

In embodiments, the exercise equipment 12 includes a seatback 114. For example, the stationary bicycle 94 includes the seatback 114. The person 14 sits on the seat 110 and the seatback 114 limits backward movement of the person 14.

The exercise equipment 12 includes an exposed surface 116. In embodiments, the exposed surface 116 is disposed at one or more of: the handle 108 of the exercise equipment 12; the seat 110 of the exercise equipment 12; the control panel 112 of the exercise equipment 12; and the seatback 114 of the exercise equipment 12. In other words, in embodiments, one or more of the handles 108, the seat 110, the control panel 112, and the seatback 114 comprises the exposed surface 116.

Each of the particular examples of the exercise equipment 12 mentioned above can comprise the exposed surface 116. In other words, in embodiments, the weight bench 34 comprises the exposed surface 116. In embodiments, the dumbbell 16 comprises the exposed surface 116. In embodiments, the kettlebell 18 comprises the exposed surface 116. In embodiments, the weight plate 22 comprises the exposed surface 116. In embodiments, the stack machine 26 comprises the exposed surface 116. In embodiments, the stationary bicycle 94 comprises the exposed surface 116. In embodiments, the treadmill 96 comprises the exposed surface 116. In embodiments, the rowing machine 104 comprises the exposed surface 116. In embodiments, the stair machine 106 comprises the exposed surface 116. In embodiments, one or more of: the barbell 20, the collar 24, the plate-loaded machine 28, the head/neck harness 30, the elastic band 32, the workout bench 36, the yoke 38, the training sled 40, the log 42, the farmer's walk handles 44, the stone 46, the keg 48, the power pin 50, the hammer 52, the stammer 54, the sandbag 56, the Bulgarian bag 58, the parallel bars 60, the pull-up and dip belt 62, the peg board 64 and the pegs 66, the pull-up bar 68, the gymnastic rings 70, the weighted vest 72, the limb weights 74, the paralletes 76, the push-up handles 78, the medicine ball 80, the Swiss ball 82, the jump rope 84, the climbing rope 86, the conditioning ropes 88, the massage roller 90, the massage ball 92, the elliptical machine 98, the glider machine 100, and the climbing machine 102 each separately comprise the exposed surface 116.

The exercise equipment 12 further includes a silane quaternary ion or salt thereof disposed on the exposed surface 116. The silane quaternary ion or salt thereof is bonded, such as covalently bonded, to the exposed surface 116. By covalently bonding to the exposed surface 116, the silane quaternary ion or salt thereof does not leach off of the exposed surface 116 and, once bonded, is difficult to wipe off. The silane quaternary ion or salt thereof destroys microbes that become disposed on the exposed surface 116, or reduces the ability of the microbes to reproduce. The silane quaternary ion or salt thereof is thought to physically destroy a microbe and is not “used up” or chemically neutralized after the destruction. The silane quaternary ion or salt thereof on the exposed surface 116 thus reduces the ability of the exercise equipment 12 to act as a vector that transmits microbes from one person 14 of the exercise equipment 12 to another person 14. Further, the silane quaternary ion or salt thereof prevents algae, mold, and other microbes from reproducing on the exposed surface 116 after the exposed surface 116 was in contact with water, such as from sweat expelled by the person 14 of the exercise equipment 12. The silane quaternary ion or salt thereof disposed on the exposed surface 116 may be particularly useful at the handle 108, the seat 110, the control panel 112, and the seatback 114 of the exercise equipment 12, which are most likely to touch the person 14 and acquire microbes from the person 14 or transmit microbes to the person 14.

In embodiments, the silane quaternary ammonium ion or salt thereof comprises one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride. In embodiments, the silane quaternary ammonium ion or salt thereof is 3-(trimethoxysilyl)propyldimethyloctadecyl ammonium ion. In embodiments, the silane quaternary ammonium ion or salt thereof is 3-(trimethoxysilyl)propyldimethyloctadecyl ammonium chloride. In embodiments, the silane quaternary ammonium ion or salt thereof is 3-(trihydroxysilyl)propyldimethyloctadecyl ammonium ion. In embodiments, the silane quaternary ammonium ion or salt is 3-(trihydroxysilyl)propyldimethyloctadecyl ammonium chloride.

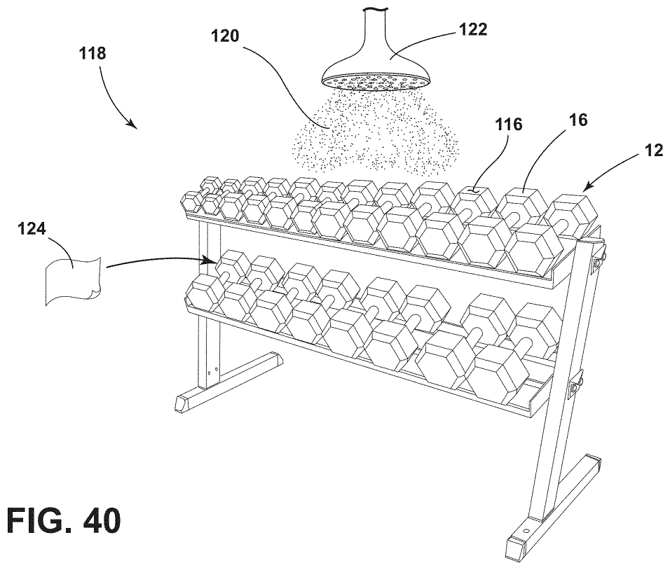

Referring now to FIG. 40, a method 118 of treating the exercise equipment 12 is herein disclosed. The method 118 includes applying a solution 120 comprising the silane quaternary ammonium ion or salt thereof to the exposed surface 116 of the exercise equipment 12. In embodiments, applying the solution 120 includes spraying the solution 120 onto the exposed surface 116 with a sprayer 122, such as an electrostatic sprayer. In other embodiments, applying the solution 120 includes wiping the exposed surface 116 with a wipe 124 that is soaked in the solution 120. In other embodiments, applying the solution 120 includes brushing the solution 120 onto the exposed surface 116. In embodiments, the exercise equipment 12 on which the solution 120 is applied (to the exposed surface 116 thereof) comprises any one or more of: the dumbbell 16, the kettlebell 18, the barbell 20, the weight plate 22, the collar 24, the stack machine 26, the plate-loaded machine 28, the head/neck harness 30, the elastic band 32, the weight bench 34, the workout bench 36, the yoke 38, the training sled 40, the log 42, the farmer's walk handles 44, the stone 46, the keg 48, the power pin 50, the hammer 52, the stammer 54, the sandbag 56, the Bulgarian bag 58, the parallel bars 60, the pull-up and dip belt 62, the peg board 64 and the pegs 66, the pull-up bar 68, the gymnastic rings 70, the weighted vest 72, the limb weights 74, the paralletes 76, the push-up handles 78, the medicine ball 80, the Swiss ball 82, the jump rope 84, the climbing rope 86, the conditioning ropes 88, the massage roller 90, the massage ball 92, the stationary bicycle 94, the treadmill 96, the elliptical machine 98, the glider machine 100, the climbing machine 102, the rowing machine 104, and the stair machine 106. In embodiments, the solution 120 is applied to the exposed surface 116 at any one or more of the control panels 112, the handle 108, the seat 110, and the seatback 114 of the exercise equipment 12. In embodiments, the solution 120 applied to the exercise equipment 12 generally includes all exposed surfaces 116.

In addition, or in the alternative, the solution 120 can be applied to individual parts of any of the exercise equipment 12 heretofore described before the individual parts are assembled into useable exercise equipment 12. For example, the solution 120 can be applied to the handle 108 before the handle 108 is assembled with other parts into the exercise equipment 12 that includes the handle 108.

The solution 120 includes a liquid component that carries the silane quaternary ammonium ion or salt to the exposed surface 116, allowing the silane quaternary ammonium ion or salt to bond to the exposed surface 116. Without being bound by theory, it is thought that the silane quaternary ammonium moiety chemically bonds to the exposed surface 116, providing long-lasting antimicrobial properties to the exercise equipment 12. The liquid component dries off leaving the silane quaternary ammonium ion or salt to be bonded to the exposed surface 116. The silane quaternary ammonium ion or salt thereof is not necessarily known for quick acting microbial reduction like a disinfectant. However, the silane quaternary ammonium ion or salt thereof provides residual long-lasting antimicrobial activity that is not consumed while destroying the microbe. In embodiments, the silane quaternary ammonium ion or salt thereof is between 0.1 percent and 10 percent by weight of the solution 120.

In embodiments, the solution 120 further comprises water as at least a portion of the liquid component. In embodiments, the solution 120 further comprises isopropyl alcohol as at least a portion of the liquid component (e.g., as a solvent). In embodiments, the isopropyl alcohol is between 30 percent to 90 percent by weight of the solution 120, such as 55 percent to 65 percent by weight of the solution 120. In embodiments, the solution 120 includes both water and isopropyl alcohol. An example solution 120 comprises (by weight) 60.0 percent isopropyl alcohol, 2.02 percent 3-(trimethoxysilyl)propyldimethyloctadecyl ammonium chloride or 3-(trihydroxysilyl)propyldimethyloctadecyl ammonium chloride, and 34.19 percent deionized water.

In embodiments, the solution 120 further comprises hydrogen peroxide. In embodiments, the hydrogen peroxide is 0.05 percent by weight to 7.5 percent by weight of the solution 120. The hydrogen peroxide operates as a disinfectant, which compliments the long-lasting antimicrobial properties of the silane quaternary ammonium ion or salt.

In embodiments, the solution 120 further comprises hypochlorous acid. In embodiments, the solution 120 comprises hypochlorous acid, a silane quaternary ammonium ion or salt thereof, and water. In embodiments, the hypochlorous acid exists in the solution 120 at a concentration of 0.1 to 1,000 ppm. In embodiments, the hypochlorous acid exists in the solution 120 at a concentration of 0.1 to 200 ppm. In embodiments, the hypochlorous acid exists in the solution 120 at a concentration of 200 to 1,000 ppm. The hypochlorous acid operates as a disinfectant, which compliments the long-lasting antimicrobial properties of the silane quaternary ammonium ion or salt.

It is to be understood that variations and modifications can be made on the aforementioned structure without departing from the concepts of the present invention, and further it is to be understood that such concepts are intended to be covered by the following claims unless these claims by their language expressly state otherwise.

The invention claimed is:

1. An exercise equipment comprising:
an exposed surface; and
a silane quaternary ammonium ion or salt thereof disposed on the exposed surface.

2. The exercise equipment of claim 1 further comprising:
a handle, wherein the handle comprises the exposed surface.

3. The exercise equipment of claim 1 further comprising:
a seat, wherein the seat comprises the exposed surface.

4. The exercise equipment of claim 1 further comprising:
a control panel, wherein the control panel comprises the exposed surface.

5. The exercise equipment of claim 1 further comprising:
a seatback, wherein the seatback comprises the exposed surface.

6. The exercise equipment of claim 1, wherein
the exercise equipment is a weight bench, and the weight bench comprises the exposed surface.

7. The exercise equipment of claim 1, wherein
the exercise equipment is a dumbbell, and the dumbbell comprises the exposed surface.

8. The exercise equipment of claim 1, wherein
the exercise equipment is a kettlebell, and the kettlebell comprises the exposed surface.

9. The exercise equipment of claim 1, wherein
the exercise equipment is a weight plate, and the weight plate comprises the exposed surface.

10. The exercise equipment of claim 1, wherein
the exercise equipment is a stack machine, and the stack machine comprises the exposed surface.

11. The exercise equipment of claim 1, wherein
the exercise equipment is a stationary bicycle, and the stationary bicycle comprises the exposed surface.

12. The exercise equipment of claim 1, wherein
the exercise equipment is a treadmill, and the treadmill comprises the exposed surface.

13. The exercise equipment of claim 1, wherein
the exercise equipment is a rowing machine, and the rowing machine comprises the exposed surface.

14. The exercise equipment of claim 1, wherein
the exercise equipment is a stair machine, and the stair machine comprises the exposed surface.

15. The exercise equipment of claim 1, wherein
the silane quaternary ammonium ion or salt thereof comprises one or more of:
3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

16. A method of treating an exercise equipment comprising:
applying a solution comprising a silane quaternary ammonium ion or salt thereof to an exposed surface of an exercise equipment.

17. The method of claim 16, wherein
the exercise equipment comprises a handle; and
the handle comprises the exposed surface.

18. The method of claim 16, wherein
the solution further comprises isopropyl alcohol.

19. The method of claim 16, wherein
the solution further comprises hydrogen peroxide.

20. The method of claim 16, wherein
the solution further comprises hypochlorous acid.
